(19) United States
(12) Patent Application Publication
(10) Pub. No.: US 2019/0041370 A1
(43) Pub. Date: Feb. 7, 2019

(54) OPTICAL SENSING MATERIALS COMPRISING METAL OXIDE NANOWIRES

(71) Applicant: United States Department of Energy, Washington, DC (US)

(72) Inventors: Pu-Xian Gao, Coventry, CT (US); Paul Ohodnicki, Allison Park, PA (US)

(21) Appl. No.: 16/043,129

(22) Filed: Jul. 23, 2018

Related U.S. Application Data
(60) Provisional application No. 62/535,517, filed on Jul. 21, 2017.

(51) Int. Cl. G01N 33/00

(52) U.S. Cl. CPC G01N 33/004; B82Y 15/00; G01N 33/005

(57) ABSTRACT

Materials, methods of making, and methods of using an apparatus for sensing. The apparatus includes an optical sensing platform; and metal oxide based nanowires incorporated into the optical sensing platform.

### CROSS-REFERENCE TO RELATED APPLICATIONS

This application claims the benefit of and priority to U.S. Provisional Application 62/535,517 filed Jul. 21, 2017, which is incorporated herein by reference in its entirety.

### STATEMENT OF GOVERNMENT SUPPORT

The United States Government has rights in this invention pursuant to the employer-employee relationship of the Government to the inventors as U.S. Department of Energy employees and site-support contractors at the National Energy Technology Laboratory. Additionally, the United States Government has rights in this invention pursuant to Grant DE-FE0011577 from the United States Government to the University of Connecticut.

### FIELD OF THE INVENTION

One or more embodiments consistent with the present disclosure relate to optical sensing materials and systems comprising metal oxide nanowires.

### BACKGROUND

Thin film and thick film metal oxide based materials are typically employed as the active layer in harsh environment chemical sensing. However, these sensing layers do not have sufficient sensitivity and chemical selectivity in many applications, which in some cases may be limited by their microstructure and the lack of a sufficiently large surface area.

Metal oxide nanowire based materials have been employed to address these weaknesses by increasing the surface area and allowing for a dimension sufficiently small relative to the so-called Debye length to result in large modifications to electronic transport properties in response to gas/solid—phase interactions at surfaces. Incorporation of functional nanoparticles including noble metals (Pd, Pt, Au) and other oxides, for example, have previously been exploited to optimize the overall sensing response through modifications to catalytic activity and/or electronic properties of the nanocomposite systems.

However, nanowire based metal oxides have only been applied previously to resistive-based and electrical-based sensing platforms which have inherent weaknesses when used in harsh environment and high temperature sensing applications. Limitations that are well known to arise include (1) prohibitive costs of electrical wiring comprised of high temperature metals such as Pt, (2) the lack of adequate and cost-effective electrical insulation for temperatures above ˜700° C., (3) high failure rates of electrical contacts in high temperature environments, and (4) safety concerns associated with electrical wiring and components in flammable gas environments, among others.

Additionally, optical based sensing platforms have a number of inherent advantages as compared to electrical-based sensors when used in high temperature and harsh environment sensing applications. Recent work has demonstrated the application of thin film and thick film metal-oxide based materials for a range of high temperature and harsh environment chemical sensing environments. However, selectivity and sensitivity in this class of sensing devices is still an area where further work is required and there is an unmet need for devices comprising advanced sensor materials. In addition, the magnitude and wavelength dependence of the response for such devices can only be tuned to within a limited extent because they are constrained by the physics of planar surfaces.

Metal oxide based nanowire materials as disclosed herein provide a number of advantages for sensor materials in chemi-resistive sensing applications including: large surface areas to enhance the degree of interaction between the analyte and the sensor material; and small nanowire diameters relative to the so-called Debye length to ensure a relatively large modulation in electrical resistivity. In other aspects nanowire surfaces may be decorated with catalytic, plasmonic, and/or other types of functional nanoparticles. In still other aspects metal oxide based nanowire materials provide the ability to engineer microstructural parameters including diameter, interconnectivity, and even heterostructuring using multiple oxides. Some illustrative embodiments of metal oxide-based nanowire materials as disclosed herein are illustrated below in FIGS. 1A-1D.

In the case of optical-based sensing platforms, additional inherent advantages of the approach disclosed herein include the ability to tailor the magnitude and sign of the optical response of the material through microstructural modifications as well as leveraging the inherent anisotropic nature of the microstructure to introduce new optical features. Such features include photonic bandgaps and resonant optical behavior at characteristic wavelengths that are associated with the specific dimensions, periodicity, orientation, and/or other microstructural aspects of the nanowire-based microstructures disclosed herein.

Methods and systems are disclosed for applying metal oxide nanowire based sensor layers to optical sensing platforms including planar thin films, planar waveguides, and optical fiber-based sensor devices. In particular preferred embodiments such materials are applied for chemical and physical sensing under high temperature and under other harsh environment conditions. Other embodiments include the full range of novel applications of metal oxide-based nanowires as the sensing material in optical-based sensor platforms.

These and other objects, aspects, and advantages of the present disclosure will become better understood with reference to the accompanying description and claims.

### SUMMARY

Embodiments of the invention relate to methods and systems for applying metal oxide nanowire based sensor layers to optical sensing platforms including planar thin films, planar waveguides, and optical fiber-based sensor devices. In particular preferred embodiments of such materials are applied for chemical and physical sensing under high temperature and under other harsh environment conditions. Other embodiments include the full range of novel applications of metal oxide-based nanowires as the sensing material in optical-based sensor platforms.

Materials, methods of making, and methods of using an apparatus for sensing. The apparatus includes an optical sensing platform; and metal oxide based nanowires incorporated into the optical sensing platform.

Materials, methods of making, and methods of using an optical sensor apparatus for sensing a gas. The apparatus includes an optical sensing platform; and metal oxide based nanowires incorporated into the optical sensing platform. In at least one embodiment where the optical sensor is effective for sensing a gas such as hydrogen or carbon monoxide, the sensor operates effectively at high temperatures between about 200° C. and about 500° C.

The following U.S. Patents and U.S. Patent Applications are incorporated herein by reference in their entirety:
US Patent Application No. 2011/0073837 to Zhou et al titled “High-performance single-crystalline n-type dopant-doped metal oxide nanowires for transparent thin film transistors and active matrix organic light-emitting diode displays”.
U.S. Pat. No. 7,235,129 to Cheng et al titled “Substrate having a zinc oxide nanowire array normal to its surface and fabrication method thereof”.
The following articles are incorporated herein by reference in their entirety: “ZnO/Perovskite core-shell nanorod array based monolithic catalysts with enhanced propane oxidation and material utilization efficiency at low temperature,” S. B. Wang, Z. Ren, W. Q. Song, Y. B. Guo, S. L. Suib, P. X. Gao, Catalysis Today, 2015, 10.1016/j.cattod.2015.03.026
“Robust 3-D Configurated Metal Oxide Nano-array based Monolithic Catalysts with Ultrahigh Materials Usage Efficiency and Catalytic Performance Tunability,” Y. B. Guo, Z. Ren, W. Xiao, C. H. Liu, H. Sharma, H. Y. Gao, A. Mhadeshwar, and P. X. Gao, Nano Energy, 2013, 2, 873-881. “Multifunctional Composite Nanostructures for Energy and Environmental Applications,” P. X. Gao, P. Shimpi, et al., Int. J. Mole. Sci., 2012, 13(6), 7393-7423.
“In-situ TPR Removal: A Generic Method for Fabricating Tubular Structure Array Devices with Mechanical and Structural Soundness, and Functional Robustness on Various Substrates,” Z. H. Zhang, H. Y. Gao, W. J. Cai, C. H. Liu, Y. B. Guo, and P. X. Gao, J. Mater. Chem., 2012, 22 (43), 23098-23105. “Synthesis, Characterization, and Photocatalytic Properties of ZnO/(La,Sr)CoO₃ Composite Nanorod Arrays,” D. L. Jian, P. X. Gao, W. J. Cai, B. S. Allimi, S. P. Alpay, Y. Ding, Z. L. Wang, C. Brooks, J. Mater. Chem., 2009, 19, 970.
“Optical and chemi-resistive sensing in extreme environments: La-doped SrTiO₃ films for Hydrogen Sensing at High Temperatures”, A. Schultz, T. D. Brown, and P. R. Ohodnicki, Journal of Physical Chemistry C 119 (11), 6211-6220 (2015).
“Optical gas sensing responses in transparent conducting oxides with large free carrier density”, P. R. Ohodnicki Jr., M. Andio, and C. Wang, Journal of Applied Physics 116, 024309 (2014).
“High Temperature Optical Sensing of Gas and Temperature Using Au-Nanoparticle Incorporated Oxides”, P. R. Ohodnicki, T. D. Brown, G. R. Holcomb, J. Tylczak, A. M. Schultz, and J. P. Baltrus, Sensors and Actuators B, 202 (31), 489-499 (2014).
“Plasmonic Nanocomposite Thin Film Enabled Fiber Optic Sensors for Simultaneous Gas and Temperature Sensing at Extreme Temperatures”, P. R. Ohodnicki et al., Nanoscale, Vol. 5 (19), 9030-9039 (2013).
“Plasmonic Transparent Conducting Metal Oxide Nanoparticles and Nanoparticle Films for Optical Sensing Applications”, P. R. Ohodnicki et al, 539 (31) 327-336 (2013).
“ZnO/Pt Nanowire Array Integrated Optical Substrates for Transmission-based Gas Sensing at Elevated Temperature” M. Zhang et al.

### BRIEF DESCRIPTION OF THE DRAWINGS

These and other features, aspects, and advantages of the multiple embodiments of the present invention will become better understood with reference to the following description, appended claims, and accompanied drawings where:

FIGS. 1A-1D depict schematic illustrations of embodiments of the disclosed technology including FIG. 1A depicts metal-oxide based nanowires, FIG. 1B depicts nanoparticle functionalized (catalysts, plasmonic, etc.) metal-oxide based nanowires, FIG. 1C depicts microstructure engineered and/or heterostructured (multiple oxide) metal-oxide based nanowires, and FIG. 1D depicts corresponding nanoparticle functionalized systems;

FIG. 2 depicts an example of a flow for synthesis of metal oxide/perovskite heterostructured nanowire arrays on solid substrates;

FIGS. 3A-3C depict typical cross-sectional view SEM images taken on the grown ZnO wire arrays, where FIG. 3A illustrates a 100 nm in diameter, 2-3 μm in length on one side of glass slide (sample 1) and FIG. 3B 1-2 μm in diameter and 4-7 μm in length on 2 sides of glass slide (sample 3) and FIG. 3C 2-3 μm in diameter and 10 μm in length uniformly on an etched optical fiber surface;

FIGS. 4A-4C depict magnification images and X-ray spectrum where FIG. 4A depicts a typical top view of a low magnification image; FIG. 4B depicts high magnification SEM images of ZnO nanowire arrays decorated with 10 nm Pt nanoparticles; and FIG. 4C depicts the corresponding energy dispersive X-ray spectrum confirming the contained elements of Zn, O and Pt in the Pt decorated ZnO nanowire arrays;

FIGS. 5A-5B depict schematic illustration of the application of one or more embodiments related to a sensing element in optical fiber based sensing platform where FIG. 5A depict transmission based sensing devices and FIG. 5B depicts reflectance based sensing devices;

FIGS. 6A-6B depict schematic illustration of the application of the proposed invention to a planar optical based sensing configuration including FIG. 6A illustrates transmission spectroscopy and FIG. 6B illustrates surface plasmon resonance based sensors in a Kretschmann configuration;

FIGS. 7A-7B depict optical sensing responses in a transmission spectroscopy configuration for two different ZnO films deposited on planar glass substrates showing responses to CO exposures of 1-20 volume % with negligible responses to varying O₂ concentration; where FIG. 7A depicts increases in transmission are illustrated for CO exposures at 300° C.; and FIG. 7B illustrates wavelength dependent responses to CO exposures are illustrated with increases in transmission for 600 nm and 1200 nm and a decrease for 2200 nm.

### DETAILED DESCRIPTION

The following description is provided to enable any person skilled in the art to use the invention and sets forth the best mode contemplated by the inventors for carrying out the invention. Various modifications, however, will remain readily apparent to those skilled in the art, since the principles of the present invention are defined herein specifically to provide nanowire based optical sensor platforms, and methods for using such materials and sensor technologies.

One or more preferred embodiments disclosed herein involve the integration of sensor materials with optical fiber based sensor platforms for harsh environment chemical sensing applications that include but are not limited to process monitoring and control in power generation systems including solid oxide fuel cells, gas turbines, and combustion systems. Other embodiments include the application of such sensors for process and emissions monitoring and control in automotive, oil and gas, aerospace, aviation, and industrial manufacturing.

In still other embodiments, disclosed metal oxide nanowire based optical sensor materials are applied in optical sensor platforms such as transmission spectroscopy, planar optical waveguides, and responsive coatings on surfaces to be monitored remotely through optical-spectroscopy based techniques. These sensing platforms may also be employed in industries as described immediately above.

It is an object of the disclosed technology to provide improved product opportunities to sensor manufacturing companies that integrate metal-oxide based nanostructure samples into optical sensing device platforms. And sensor performance advantages to users of the fabricated sensors.

One or more preferred embodiments relate to combinations of metal oxide based nanowire sensing materials with optical sensing platforms such as optical fiber sensors. The resulting systems and devices combine the advantages of enhanced selectivity and sensitivity achieved with this class of metal oxide based materials with the inherent advantages of optical based systems for harsh environment sensing.

Also disclosed herein are new functionalities not previously attainable that can be realized through the combination of these two components including: (1) plasmonic responses in metal nanowire-based sensor material systems integrated with plasmonically active metallic nanoparticles such as Ag, Au, etc., (2) exploitation of ambient gas atmosphere dependent photonic bandgap effects for metal oxide nanowire based systems with periodicity in size, spacing, and density, and (3) enhanced sensitivity of extinction by the sensing material to the real part of the refractive index associated with increased light scattering in metal oxide based nanowire sensor materials. It is an object of these new functionalities to enable development of optical based sensors with improved sensitivity and selectivity. It is a further object to provide additional sensing modalities not previously possible in devices for use in high temperature and other harsh environmental conditions.

In the case of optical-based sensing platforms, additional inherent advantages of the approach disclosed herein include the ability to tailor the magnitude and sign of the optical response of the material through microstructural modifications as well as leveraging the inherent anisotropic nature of the microstructure to introduce new optical features. Such features include photonic bandgaps and resonant optical behavior at characteristic wavelengths that are associated with the specific dimensions, periodicity, orientation, and/or other microstructural aspects of the nanowire-based microstructures disclosed herein.

Typical metal-oxide materials that may be utilized in the composition of one or more of the nanowires include: standard binary oxides such as SnO₂, CeO₂, Ga₂O₃, TiO₂, ZnO, WO₃, FeₓO_{y}, CoₓO_{y} and NixO_{y}; doped variants of the previously mentioned binary oxides such as Al-doped ZnO and Nb-doped TiO₂; perovskite oxides such as LaCoO₃, LaFeO₃, LaMnO₃, LaNiO₃, SrTiO₃, and SrFeO₃, and doped variants of perovskite oxides such as Sr-doped LaMnO₃ (LSM) and Sr,Fe-doped LaCoO₃ (LSCF).

Typical materials that may comprise the functionalizing nanoparticles disclosed herein include: traditional metal catalysts such as Au, Pd, Pt, Cu, and Rh; oxide-based catalysts such as FeₓO_{y}, NiₓO_{y}, CrₓO_{y}, and CoₓO_{y}; perovskite oxide based catalysts such as Sr-doped LaFeO₃ and Sr, Fe-doped LaCoO₃, and plasmonic nanoparticles such as Au, Ag, Sn-doped In₂O₃, and Al-doped ZnO.

One or more embodiments of metal-oxide based nanowires for optical sensing retains inherent advantages of metal oxide based thin films and thick films including chemically responsive properties and the ability to tailor properties, selectivity, and sensitivity through doping, incorporation of functional nanoparticles such as catalysts and plasmonic materials, and stability under high temperature and harsh environment conditions.

Disclosed herein are advantages of the described materials including the ability to tune the optical response of the material through microstructural modifications of the following type:

Tailoring the degree of light scattering by nanowire length, diameter, and density allowing for an enhanced sensitivity to changes in the real part of a refractive index of the material.

The ability to modify the propagation of light through films of aligned nanowires through imparting geometrical periodicity in the size and spacing including photonic bandgaps and waveguiding behavior.

Utilization of multiple oxide materials to form heterostructured oxides and/or junctions.

Incorporation of functional nanoparticles onto or into the metal oxide based nanowire structure to incorporate new electrical or optical properties and/or to enhance the sensing response through catalytic activity.

### EXAMPLES

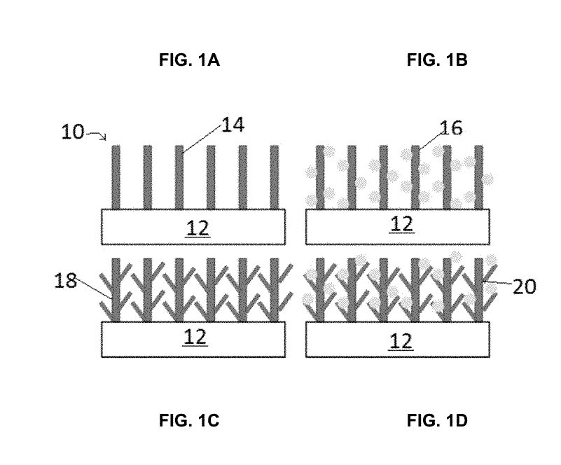

Disclosed herein are systems and devices comprising optical based sensing platforms integrated with metal oxide nanowire based sensing layers as schematically illustrated in FIGS. 1A-1D. The integration strategy is based on synthetic methods combining vapor phase deposition and solution deposition. Through this strategy, various metal oxide nanowire arrays 10 may be grown directly onto surfaces 12, including both planar and three-dimensional curved surfaces such as Si and glass wafers, and etched optical fibers that may be used in both transmission and reflectance optical interrogation modes. Preferred materials for metal oxide nanowires and for functionalizing nanoparticles are described herein.

FIGS. 1A-1D further depict schematic illustrations of embodiments of the disclosed technology including FIG. 1A which illustrate metal-oxide based nanowires 14, FIG. 1B illustrates nanoparticle functionalized (catalytic, plasmonic, etc.) metal-oxide based nanowires 16, FIG. 1C illustrates microstructure engineered and/or heterostructured (multiple oxide) metal-oxide based nanowires 18, and FIG. 1D illustrates corresponding nanoparticle functionalized systems 20.

### Example—Synthesis of Metal Oxide/Perovskite Heterostructured Nanowire Arrays

The disclosed technology is further illustrated by the following general description of method choices and logistics in material processing.

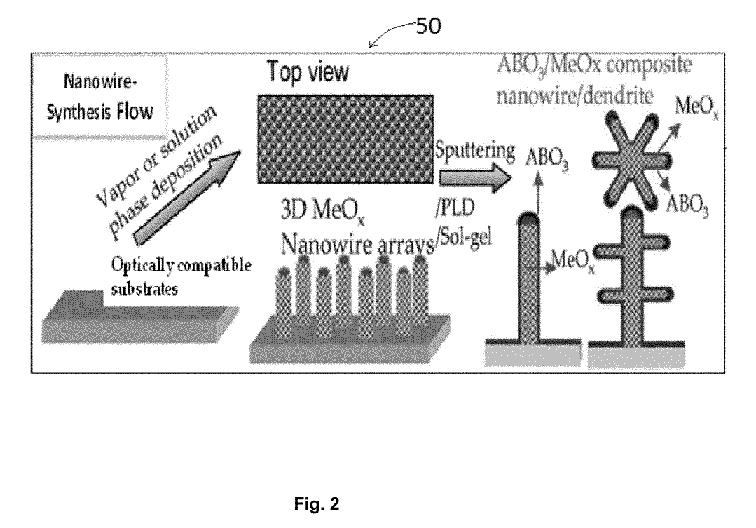

FIG. 2: depicts an example of a flow diagram 50 for synthesis of metal oxide/perovskite heterostructured nanowire arrays on solid substrates. As illustrated in FIG. 2, the synthesis of metal oxide/perovskite heterostructured nanowire arrays, a combination method of solution phase and vapor phase deposition is utilized for the growth of metal oxide nanowire based materials on planar and fiber optical substrates. Specifically, hydrothermal synthesis or thermal evaporation is employed to grow metal oxide nanowire arrays on optically compatible substrates such as Si and glass, and an atomic layer deposition, pulsed laser deposition, or magnetron sputtering, or sol-gel washcoating process may be used to form continuous or mesoporous perovskite nanoparticle decoration on metal oxide nanowire surfaces.

As an exemplary set-up for metal oxide nanowire array growth, the thermal evaporation apparatus consists of a horizontal high temperature tube furnace, an alumina tube, a rotary pump system and a gas controlling system. Taking ZnO nanowire array as an example, commercial ZnO, and graphite with certain weight ratio were fully mixed by grounding the powder mixture for 15 minutes and then used as the source material. The source material was loaded on an alumina boat and positioned at the center of the alumina tube. After evacuating the tube to 2×10⁻³ Torr, thermal evaporation was conducted at high temperature for one hour under certain pressure and Ar carrier gas flow with certain rate (standard cubic centimeters per minute, sccm). The prepared nanostructures were collected in downstream low temperature regions in specific deposition optically compatible substrates. During the synthesis, some important parameters in the chamber such as the pressure, temperature gradient, gas flow rate, and especially the source materials composition are controlled for producing nanowires with ultra-high surface area. To control the orientation, dimensions and spatial distribution of the metal oxide nanowire arrays, the design and selection of substrates, seeding materials and processing parameters are critical for both vapor and solution growth processes.

For the deposition of nanoparticles of perovskite oxides such as (La, Sr)MO₃ (M=Co and Fe) and noble metals such as Pt and Pd, both solution phase and vapor phase deposition methods can be used. The solution phase approaches include sol gel and hydrothermal synthesis. The vapor phase approaches include magnetron sputtering and pulsed laser deposition (PLD) methods. As an example, PLD be used to fabricate the perovskite nanoparticle layer of nanowire arrays. In this exemplary process, a laser beam with a certain pulse and energy intensity is used as the heating source to shoot and vaporize the perovskite target surface, and the carrier plasma plum transfers the vaporized source onto the substrate with as-grown metal oxide nanowire arrays from the thermal evaporation process. Then a short period of fast deposition of perovskite nanoparticles produces the perovskite layer surrounding the ZnO nanowire array core.

Using ZnO nanowire arrays in conjunction with the secondary and ternary components such as Pt and LaMnO₃, the disclosed synthetic methods are further described below for the growth of 1) ZnO nanowire arrays on glass substrates; 2) Pt and LaMnO₃ nanoparticles decorated ZnO nanowire arrays on ITO coated glass substrates.

### Example—ZnO Nanowire Arrays on Glass Slide and Pre-Etched Optical Fiber Sample Preparation

Firstly, a thin layer of ZnO layer was deposited on glass substrates. For Sample 1, 30 nm thick ZnO seed layer was deposited by a sputtering method. For Sample 3 and pre-etched optical fiber surface sample, a solution was prepared by 20 mM zinc acetate dihydrate [Zn(CH₃COO)₂,2H₂O] and pure ethanol, and wash coating and dry for 10 times. There is only one side had been coated with ZnO on Sample 2, and double sides coated with ZnO on Sample 3. All the three samples were annealed at 350° C. for 2 h. Secondly, the ZnO nanorod arrays were synthesized by hydrothermal method. All the three samples were simply dipped into 25 mM zinc nitrate hexahydrate [Zn(NO₃)₂.6H₂O] and 25 mM hexamethylenetetramine (HMTA, C₆H₁₂N₄) solution for 5 h at 90° C. water bath. After 5 h hydrothermal synthesis, samples were ultrasonic cleaned by pure ethanol for 5 min and dried in furnace at atmosphere.

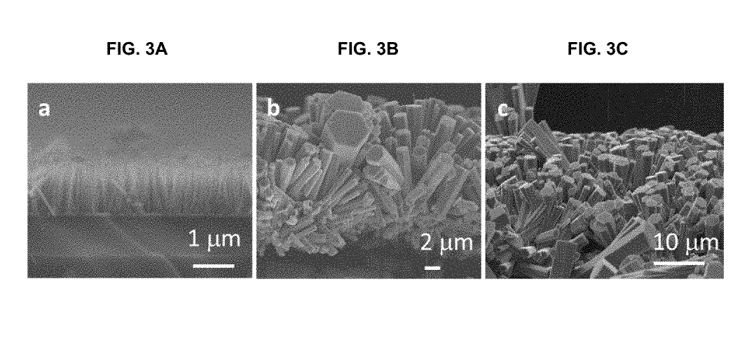

FIGS. 3A-3C depict typical cross-sectional view SEM images taken on the grown ZnO wire arrays wherein FIG. 3A depicts a 100 nm in diameter, 2-3 μm in length on one side of glass slide; FIG. 3B depicts 1-2 μm in diameter and 4-7 μm in length on 2 sides of glass slide; and FIG. 3C depicts 2-3 μm in diameter and 10 μm in length uniformly on an etched optical fiber surface.

### Example—Preparation of ZnO/Pt (LaMnO₃) Nanorod Array on ITO Glass

Firstly, ITO glass substrates were washed in acetone and ethanol sequentially to remove surface contaminant. A 30 nm thick ZnO seed layer was deposited using RF magnetron sputtering. After the seeding process, the substrates were annealed at 350° C. for 3 h to enhance the crystallinity of seed layer. ZnO nanorod array growth was performed using a continuous flow method developed in house. The growth solution of zinc acetate and HMT mixture with concentration of 12.5 mM was used and the flow rate was maintained at 4 ml/min. The substrates were suspended in the growth solution with seeded side towards bottom and the reactor was kept in oil bath at 90° C. After 8 h, the substrates were washed in ethanol and dried for next step.

Secondly, Pt nanoparticles were deposited on ZnO nanorod array using sputtering method. The ZnO nanorod array based ITO glasses were horizontally placed in sputtering machine. Pt nanoparticles coating with thickness of 10 nm was uniformly deposited on ZnO nanorod array.

Thirdly, for LaMnO₃ deposition on ZnO nanorod array, first colloidal solution was prepared by dissolving equal amount of 0.0024 mole La(NO₃)₃.6H₂O and Mn(NO₃)₂.4H₂O in 20 ml 2-Ethoxyethanol. After that, 0.05 g PVP (Mw 55000) and 0.2 ml diethanolamine were added into solution under vigorous stir. Then the solution was aged in air for 72 h. The LaMn₃O₄ deposition was obtained using dip-coating method. The substrate was submerged in colloidal solution and vertically dragged out from the solution. Then the substrate was placed horizontally and dried on hot plate at 60° C. This process was repeated for multiple cycles until desired amount of material was deposited on the ZnO nanorod array. After that, the substrate was annealed in furnace at 350° C. for 1 h.

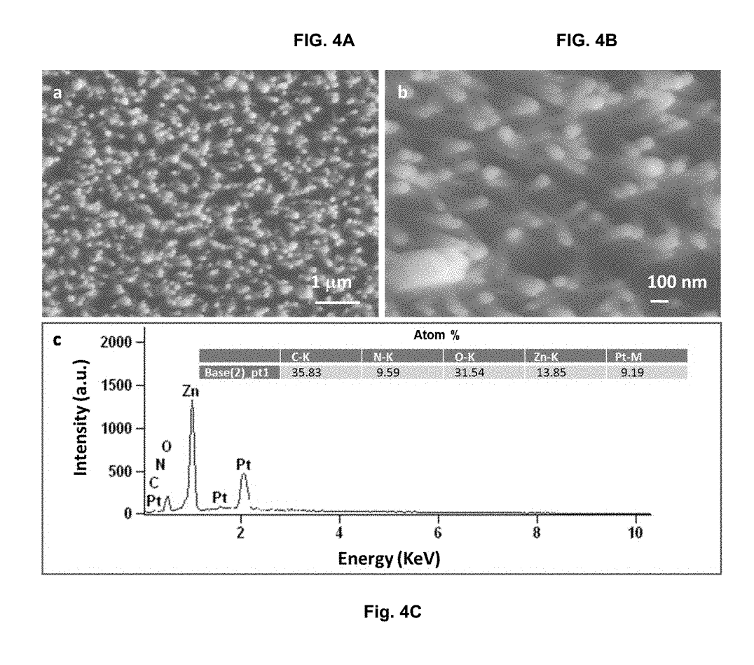

FIGS. 4A-4B depict typical top view of arrays where FIG. 4A depicts a low magnification and FIG. 4B depicts high magnification SEM images of ZnO nanowire arrays decorated with 10 nm Pt nanoparticles; and the corresponding energy dispersive X-ray spectrum confirming the contained elements of Zn, O and Pt in the Pt decorated ZnO nanowire arrays.

### Example—Application of Sensing Layers to Form Sensing Elements in Optical Fiber Based Sensing Platforms

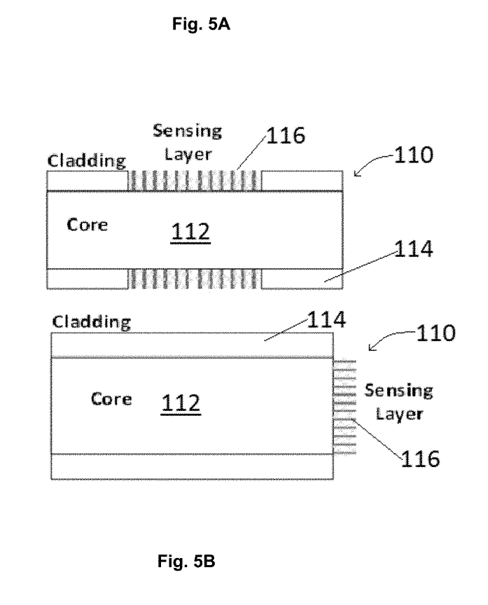

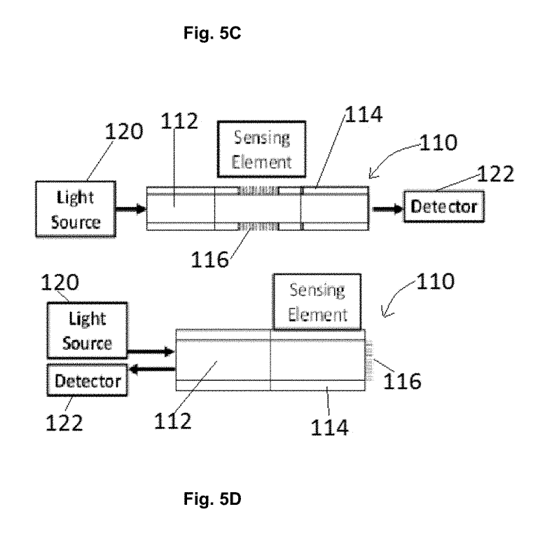

In FIGS. 5A-5D depict the application of the sensing layers to form sensing elements in optical fiber based sensing platforms for both transmission based (See FIGS. 5A and 5C) and reflectance based sensing devices (See FIGS. 5B and 5D).

More specifically, FIGS. 5A-5B depict schematic illustration of the application of the proposed invention to a sensing element in optical fiber based sensing platform in FIG. 5A depict transmission based sensing devices 110 and FIG. 5B depicts reflectance based sensing devices 110. The core 112 is typically comprised of an optically transparent material such as silica or sapphire and the cladding 114 is a similarly transparent material with a slightly lower index of refraction. The sensing layer 116 is comprised of a metal oxide nanowire based material such as the types illustrated as in FIGS. 1A-1D above. FIGS. 5C and 5D depict a schematic of the full fiber optic based sensor illustrated for the transmission and reflectance based sensors, respectively. FIGS. 5C and 5D further depict a light source 120 and light detector 122 in communication with the devices 110.

### Example—Application of Sensing Layers in Planar Optical Based Sensing Configurations

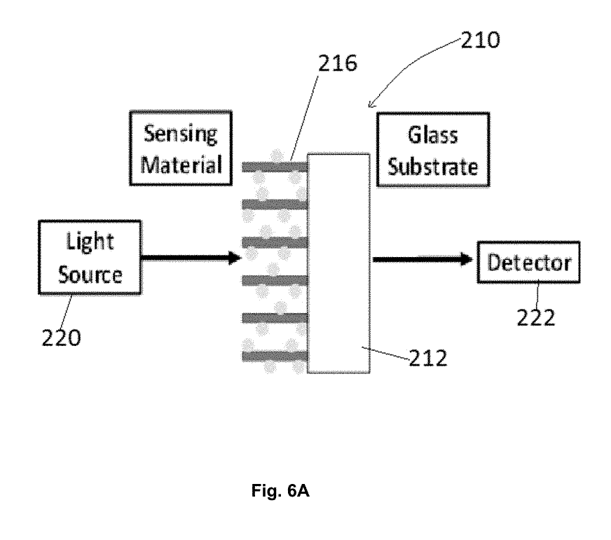

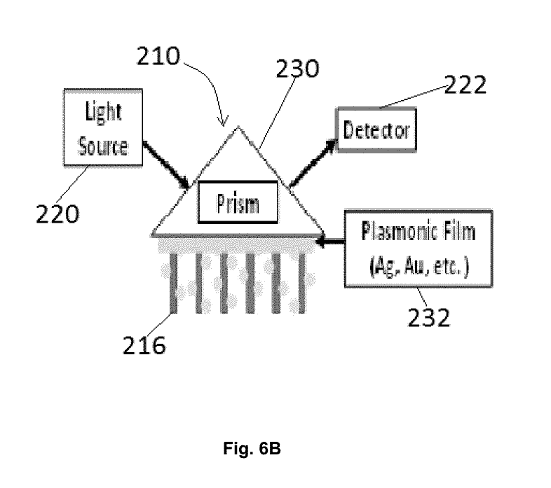

In addition to optical fiber based sensing platforms, metal oxide nanowire based sensor materials can also be integrated with planar thin film based optical sensor platforms such as transmission spectroscopy and surface plasmon resonance sensors which are interrogated through direct transmission or reflectance spectroscopy. Examples of these two types of configurations are illustrated schematically in FIGS. 6A-6B.

FIGS. 6A-6B depict schematic illustration of the application of the proposed invention to a planar optical based sensing configuration 210 including FIG. 6A which illustrate transmission spectroscopy 210 and FIG. 6B which illustrate surface plasmon esonance based sensors in a Kretschmann configuration 210.

The core 212 is typically comprised of an optically transparent material such as silica or sapphire. FIG. 6B depicts the core comprises a prism 230. The sensing layer 216 is comprised of a metal oxide nanowire based material such as the types illustrated as in FIGS. 1A-1D above. FIGS. 6A and 6B further depict a light source 220 and light detector 222 in communication with the devices 210.

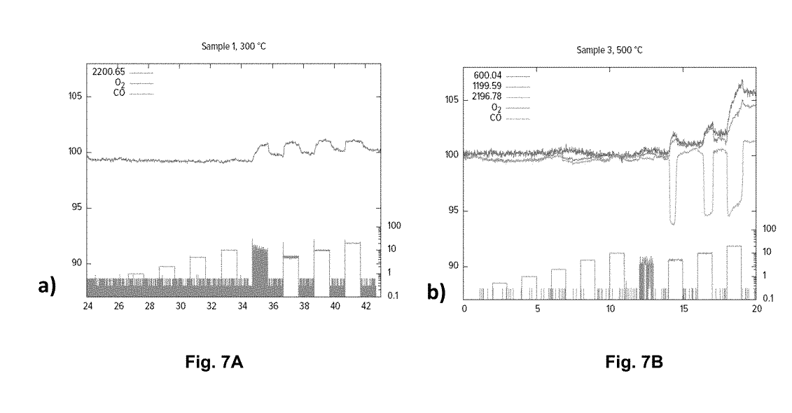

In all cases above, the measured response of the sensor configuration 210 will depend sensitively upon the composition of the metal oxide based nanowire sensing layer as well as detailed microstructure. As one example to demonstrate a proof of concept for the disclosed sensing approach, ZnO-based nanowire samples were deposited on glass substrates and exposed to a range of gas atmospheres at temperatures ranging from room temperature up to 500° C. During the exposure tests, optical film transmittance was measured in the visible and near-infrared wavelength ranges. Example results from these experiments for two different samples are presented below in FIG. 7 illustrating clear responses to reducing gas exposures of CO but negligible responses to O₂, particularly at elevated temperatures approaching bout about 200-500° C., specifically 300-500° C.

FIGS. 7A-7B depict optical sensing responses in a transmission spectroscopy configuration for two different ZnO films deposited on planar glass substrates showing responses to CO exposures of 1-20 volume % with negligible responses to varying O₂ concentration. In FIG. 7A, increases in transmission are illustrated for CO exposures at 300° C. In FIG. 7B, the wavelength dependent responses to CO exposures are illustrated with increases in transmission for 600 nm and 1200 nm and a decrease for 2200 nm.

### Example—ZnO/Pt Nanowire Array Integrated Optical Substrates for Transmission-based Gas Sensing at Elevated Temperature

Disclosed herein are integrated ZnO nanowire arrays integrated with pre-etched cylindrical optical fiber to combine the metal oxide based nanowires with optical fiber as an elevated temperature chemical sensor platform. Through a dip coating method, Pt nanoparticles have been well dispersed as sensitizers onto ZnO nanowire arrays as grown on the etched optical fiber. The ZnO/Pt nanowire integrated optical fiber has been implemented as an effective sensor to various gases such as H at 350° C. using transmission optical mode, particularly upon 500 nm, 650 nm and 800 nm reference light illumination. This successful integration of nanowire array materials and optical fiber sensing platform combines the advantages of enhanced selectivity and sensitivity achieved in this class of materials with the inherent advantages of optical based systems for harsh environment sensing as noted above, which opens a potential avenue for highly sensitive and selective optical sensors for high temperature applications.

While the invention has been described with reference to preferred embodiments, it will be understood by those skilled in the art that various changes may be made and equivalents may be substituted for the elements thereof without departing from the scope of the invention. In addition, many modifications may be made to adapt the teaching of the invention to particular use, application, manufacturing conditions, use conditions, composition, medium, size, and/or materials without departing from the essential scope and spirit of the invention. Therefore, it is intended that the invention not be limited to the particular embodiments and best mode contemplated for carrying out this invention as described herein.

While various aspects and embodiments have been disclosed herein, other aspects and embodiments will be apparent to those skilled in the art. The various aspects and embodiments disclosed herein are for purposes of illustration and are not intended to be limiting of the true scope of the invention disclosed herein. It is also to be understood that the terminology used herein is for the purpose of describing particular embodiments only, and is not intended to be limiting. Since many modifications, variations, and changes in detail can be made to the described examples, it is intended that all matters in the preceding description and shown in the accompanying figures be interpreted as illustrative and not in a limiting sense.

Chemical compounds are described using standard nomenclature. For example, any position not substituted by any indicated group is understood to have its valency filled by a bond as indicated, or a by hydrogen atom.

All ranges disclosed herein are inclusive of the endpoints, and the endpoints are independently combinable with each other. Each range disclosed herein constitutes a disclosure of any point or sub-range lying within the disclosed range.

Having described the basic concept of the embodiments, it will be apparent to those skilled in the art that the foregoing detailed disclosure is intended to be presented by way of example. Accordingly, these terms should be interpreted as indicating that insubstantial or inconsequential modifications or alterations and various improvements of the subject matter described and claimed are considered to be within the scope of the spirited embodiments as recited in the appended claims. Additionally, the recited order of the elements or sequences, or the use of numbers, letters or other designations therefor, is not intended to limit the claimed processes to any order except as may be specified. All ranges disclosed herein also encompass any and all possible sub-ranges and combinations of sub-ranges thereof. Any listed range is easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as up to, at least, greater than, less than, and the like refer to ranges which are subsequently broken down into sub-ranges as discussed above. As utilized herein, the terms “about,” “substantially,” and other similar terms are intended to have a broad meaning in conjunction with the common and accepted usage by those having ordinary skill in the art to which the subject matter of this disclosure pertains. As utilized herein, the term “approximately equal to” shall carry the meaning of being within 15, 10, 5, 4, 3, 2, or 1 percent of the subject measurement, item, unit, or concentration, with preference given to the percent variance. It should be understood by those of skill in the art who review this disclosure that these terms are intended to allow a description of certain features described and claimed without restricting the scope of these features to the exact numerical ranges provided. Accordingly, the embodiments are limited only by the following claims and equivalents thereto. All publications and patent documents cited in this application are incorporated by reference in their entirety for all purposes to the same extent as if each individual publication or patent document were so individually denoted.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

We claim:

1. An apparatus for sensing, the apparatus comprising:
an optical sensing platform; and
metal oxide based nanowires incorporated into the optical sensing platform.

2. The apparatus of claim 1 wherein the optical sensing platform comprises an optically compatible substrate.

3. The apparatus of claim 1 wherein the nanowires are functionalized with nanoparticles.

4. The apparatus of claim 1 wherein the nanowires comprise microstructure engineered metal oxide based nanowires.

5. The apparatus of claim 4 wherein the microstructure engineered metal oxide based nanowires are functionalized with nanoparticles.

6. The apparatus of claim 1 wherein the nanowires comprise heterostructured (multiple oxide) metal oxide based nanowires.

7. The apparatus of claim 6 wherein the heterostructured (multiple oxide) metal oxide based nanowires are functionalized with nanoparticles.

8. The apparatus of claim 1 wherein the nanowires comprise microstructure engineered metal oxide based nanowires and heterostructured (multiple oxide) metal oxide based nanowires.

9. The apparatus of claim 8 wherein at least one of the microstructure engineered metal oxide based nanowires and heterostructured (multiple oxide) metal oxide based nanowires are functionalized with nanoparticles.

10. The apparatus of claim 1 wherein the optical sensing platform is selected from a group comprising silicon, glass, ITO glass, optical fiber and etched optical fiber.

11. The apparatus of claim 2 wherein the optically compatible substrate is selected from a group comprising silicon, glass, ITO glass, optical fiber, or etched optical fiber.

12. The apparatus of claim 1 wherein the optical sensing platform comprises a planar surface or a curved surface.

13. The apparatus of claim 1 wherein the metal oxide based nanowires is selected from group comprising standard binary oxides; doped variants of standard binary oxides, perovskite oxides, doped variants of perovskite oxides, and combinations thereof.

14. The apparatus of claim 13 wherein the standard binary oxides are selected from the group comprising SnO₂, CeO₂, Ga₂O₃, TiO₂, ZnO, WO₃, FeₓO_{y}, CoₓO_{y} and NixO_{y}; the doped variants of standard binary oxides are selected from the group comprising Al-doped ZnO and Nb-doped TiO₂; the perovskite oxides are selected from the group comprising LaCoO₃, LaFeO₃, LaMnO₃, LaNiO₃, SrTiO₃, and SrFeO₃; and the doped variants of perovskite oxides are selected from the group comprising Sr-doped LaMnO₃ (LSM) and Sr, Fe-doped LaCoO₃ (LSCF); and combinations thereof.

15. The apparatus of claim 3 wherein the functionalized nanoparticles are selected from the group comprising traditional metal catalysts, oxide-based catalysts, perovskite oxide based catalysts, plasmonic nanoparticles and combinations thereof.

16. The apparatus of claim 15 wherein the traditional metal catalysts are selected from the group comprising Au, Pd, Pt, Cu, and Rh; the oxide-based catalysts are selected from the group comprising FeₓO_{y}, NiₓO_{y}, CrₓO_{y}, and CoₓO_{y}; the perovskite oxide based catalysts are selected from the group comprising Sr-doped LaFeO₃ and Sr,Fe-doped LaCoO₃, and plasmonic nanoparticles are selected from the group comprising Au, Ag, Sn-doped In₂O₃, and Al-doped ZnO, and combinations thereof.

17. The apparatus of claim 1 wherein the metal oxide nanowires comprise ZnO and the optical sensing platform comprises glass.

18. The apparatus claim 1 wherein the metal oxide nanowires comprise ZnO and the optical sensing platform comprises an etched optical fiber surface.

19. The apparatus of claim 3 wherein the metal oxide nanowires comprise ZnO, the optical sensing platform is selected from the group comprising ITO glass and an etched optical fiber surface and the nanowires are functionalized with Pt nanoparticles.

20. An optical sensor apparatus for sensing a gas, the apparatus comprising:
an optical sensing platform; and
metal oxide based nanowires incorporated into the optical sensing platform, wherein the optical sensor is effective for sensing a gas such as hydrogen or carbon monoxide, and further wherein the sensor operates effectively at high temperatures between about 200° C. and about 500° C.
